(19) United States
(12) Patent Application Publication
(10) Pub. No.: US 2010/0179517 A1
(43) Pub. Date: Jul. 15, 2010

(54) CATHETER

(76) Inventor: Jiro TAKASHIMA, Houston, TX (US)

Correspondence Address: Andrew W. Chu, PC, P. O. Box 2925, Houston, TX, 77252-2925, US

(21) Appl. No.: 12/684,806

(22) Filed: Jan. 8, 2010

Related U.S. Application Data
(60) Provisional application No. 61/143,632, filed on Jan. 9, 2009.

(51) Int. Cl. A61M 27/00

(52) U.S. Cl. 604/544

(57) ABSTRACT

The catheter of the present invention includes a first end with a rounded top, a second end, and a plurality of segments between the first end and the second end. The catheter may also include a connection to a medical device. Each segment has a first tapered surface with a diameter increasing from a first edge to the tapered surface edge and second tapered surface with a diameter decreasing from the tapered surface edge to a second edge. The taper surface edge has the widest diameter of the segment. The widest section of the segment is approximately between 4 mm and 6 mm inclusive for the urethral catheter. A maximum length of the segment is approximately 1.5 times the length of the diameter of the segment at the tapered surface edge. The length of the second tapered surface is greater than the length of the first tapered surface.

### RELATED U.S. APPLICATIONS

The present application claims priority under U.S. Code Section 119(e) from a provisional patent application, U.S. patent application Ser. No. 61/143632, filed on 9 Jan. 2009 and entitled “CATHETER”.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention relates to a generally cylindrical device capable of being retained within a human body cavity. More particularly, the present invention relates to self-retaining catheters without a stop means.

2. Description of Related Art Including Information Disclosed Under 37 CFR 1.97 and 37 CFR 1.98

A catheter is defined as a tubular instrument used to allow fluid to pass from or into a body cavity. Catheters are commonly used to drain urine from the urinary bladder. However, catheters are also used for drug and intravenous fluid delivery, angioplasty, and in the case of a Swan-Ganz catheter, the direct measurement of blood pressure in a vein or artery. This is not an exhaustive list, and the various types of catheters are abundant as are their uses.

A problem commonly associated with catheters is retention failure. Retention failure occurs when the catheter fails to remain at the desired location, and this can lead to loss of catheter function and exacerbation of the underlying problem that necessitated a catheter, as well as other problems such as infection, contamination, and discomfort. When catheter retention failure occurs, reinsertion is a typical response. Reinsertion can increase the probability of infection and trauma to the body cavity. Recurring episodes of retention failure and resulting reinsertion can diminish a patient's willingness to seek medical help for their underlying problem.

When a traditional cylindrical catheter is inserted into the human body cavity, the downward force created by peristaltic movement inside the human body cavity wall will continuously move the catheter towards the insertion point and gradually expel the catheter. Therefore most catheters provide a stop means such as a balloon at the first inserted end of the catheter to prevent the peristaltic movement within a body cavity from expelling the catheter.

One potential solution to the retention failure problem is the use of a balloon-tip catheter, which upon inflation inside a body cavity or lumen acts to resist peristalsis, as well as other physiological factors that contribute to retention failure and catheter expulsion from a body cavity. An example of a balloon-tip catheter is the Foley catheter. However, significant problems can arise with use of a Foley catheter. First, upon inflation the balloon may rupture. This may require surgery to repair any internal damage as well as remove the ruptured balloon fragments. Second, the balloon may be inadvertently inflated before reaching the inflation destination. This can be very painful for the user and require invasive techniques to withdraw the catheter.

Another potential solution to the catheter retention problem is to have “screw-type threads” on the exterior surface of the catheter. The catheter can be securely inserted into a body cavity by turning the device. However, major problems and complications can arise if the catheter is removed improperly. For example, if the catheter is accidentally pulled out suddenly, the body cavity can suffer devastating injury.

A third potential remedy for retention failure problem is external protrusions on the exterior surface of the catheter. These protrusions interact with the walls of the body cavity and act as anchors to prevent retention failure. However, the use of anchors can lead to irritation of the body cavity and user discomfort.

U.S. Pat. No. 5,964,732 issued to Willard on Oct. 12, 1999, claims methods of positioning a catheter within the urethra. Willard also generally describes that overcoming hydraulic forces acting to expel the catheter can be accomplished by compressive forces generated by the urethral wall acting on the longitudinal surface of the catheter. Willard also describes that a combination of surface projections along with the longitudinal surface area may offset the hydraulic and physiological forces that act to remove the catheter from the urethra. Willard generally states that retention can be achieved if the sum of forces between the urethra and the body of the catheter exceed the hydraulic and physiological forces acting to expel the catheter.

U.S. Pat. No. 5,971,967 issued to Willard on Oct. 26, 1999, describes a urethral catheter having one or more tapered anchors located on the external surface of the device. The anchors form partial spiral helices. Willard claims this conformation overcomes deficiencies in the prior art, namely, that a continuous helical surface provides a shunt pathway for urine.

It is an object of the present invention to provide a self-retaining catheter that remains within a human body cavity without a utilizing traditional stop means such as a balloon.

It is a further object of the present invention to provide a catheter that utilizes the lateral pressure of a body cavity to retain the catheter within the body cavity.

It is a further object of the present invention to provide a catheter that is easy to use, inexpensive, and easy to manufacture.

These and other objects and advantages of the present invention will become apparent from a reading of the attached specification and appended claims.

### SUMMARY OF THE INVENTION

A self-retaining catheter comprises a first end and a second end, and a plurality of segments between the first end and the second end. Each segment has a first tapered surface with a diameter increasing from a first edge to a tapered section edge and a second tapered surface with a diameter decreasing from the tapered surface edge to a second edge. The length of the first tapered surface is smaller than the length of the second tapered surface.

### BRIEF DESCRIPTION OF THE DRAWINGS

FIG. 1 is a side elevational view of a catheter 10 of the present invention.

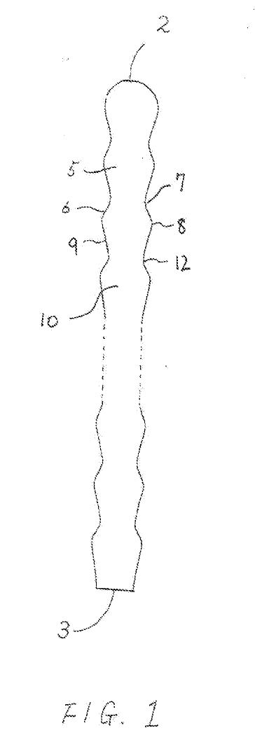

### DETAILED DESCRIPTION OF THE DRAWINGS

A catheter 10 having a first end 2, a second end 3, and a plurality of segments 5 between the first end 2 and the second end 3. Each segment 5 has a first tapered surface 6 with a diameter increasing from a first edge 7 to a tapered surface edge 8 and a second tapered surface 9 with a diameter decreasing from said tapered surface edge 8 to a second edge 12.

The length of the second tapered surface 9 is greater than the length of the first tapered surface 6.

In a urinary catheter embodiment, the maximum diameter of the tapered surface edge 8 is approximately 8 mm, and an optimum length of each segment 5 for the urinary catheter is an equal or smaller length of the diameter of the tapered surface edge 8. A maximum length of the segment 5 for the urinary catheter is approximately 1.5 times the diameter of the tapered surface edge 8.

Other embodiments of this self-retaining catheter 10 may be used for other tubular cavities of the human body, from the vascular system to the digestive system. Thus, the diameter of the self-retaining catheter 10 can vary widely, from less than 1 mm to as large as 25 mm, depending on the body cavity.

The present invention works by inserting the catheter 10 into a body cavity. Peristaltic movement in the body cavity acts to expel the catheter. In the prior art, the cylindrical tube of the catheter is expelled as a foreign object unless a utilized. In the present invention, no stop means is provided; rather, the lateral pressure and the expelling downward peristaltic movement of the body cavity acting on the first tapered surface generates a thrust toward the second end, while the lateral pressure of the body cavity acting on the second tapered surface generates a thrust towards the first end. The invention provides a sufficient second tapered surface 9 of each segment 5, having a total surface area greater than the total surface area of the first tapered surface 6. Therefore, the summation of the lateral pressure of the body cavity acting on the first and second tapered surfaces 6 and 9 generates a net thrust on the catheter towards the first end 2. The net thrust generated by the lateral pressure is sufficient to overcome the effect of the peristaltic motion that would eventually expel a traditional catheter.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. A catheter, maintained within a body cavity and interactive with said body cavity, said catheter comprising:
a first end;
a second end; and
a plurality of segments positioned between said first end and said second end, each segment having a first tapered surface with a diameter increasing from a first edge to a tapered surface edge and a second tapered surface with a diameter decreasing from the tapered surface edge to a second edge, said tapered surface edge having a widest diameter of the segment, the length of said first tapered surface being smaller than the length of said second tapered surface.

2. The catheter of claim 1, wherein each segment has a maximum length 2 times a diameter at an edge of said tapered surface.

3. The catheter of claim 1, wherein each segment has a widest diameter at said edge of said tapered surface, each segment having a length ranging between 4 mm and 8 mm inclusive for a urinary catheter.
